(19) United States
(12) Patent Application Publication
(10) Pub. No.: US 2023/0277302 A1
(43) Pub. Date: Sep. 7, 2023

(54) Haptic Management for Surgical Implants

(71) Applicant: Alcon Inc., Fribourg (CH)

(72) Inventors: Yinghui Wu, Cedar Hill, TX (US); Harlen Hoang, Fort Worth, TX (US); Nerea Garcia Ramila, Arlington, TX (US); Sudarshan B. Singh, Euless, TX (US); R. Mitchell Sherry, Fort Worth, TX (US)

(21) Appl. No.: 18/175,742

(22) Filed: Feb. 28, 2023

Related U.S. Application Data
(60) Provisional application No. 63/268,704, filed on Mar. 1, 2022.

(51) Int. Cl. A61F 2/16

(52) U.S. Cl. CPC A61F 2/167; A61F 2002/1681

(57) ABSTRACT

An apparatus for folding an intraocular lens or other implant before inserting it into an eye in various locations using variable surgical techniques. Some embodiments may comprise or consist essentially of a haptic folding mechanism configured to fold one or more haptics onto the top of an optic prior to the optic being folded into a nozzle. In some embodiments, a leading haptic lifter or lifting mechanism can be configured to elevate and constrain a leading haptic during implant delivery. This leading haptic folding mechanism can passively lift the leading haptic onto the top of the optic.

### TECHNICAL FIELD

The invention set forth in the appended claims relates generally to eye surgery, including, without limitation, systems, apparatuses, and methods for inserting an implant into an eye.

### BACKGROUND

The human eye can suffer a number of maladies causing mild deterioration to complete loss of vision. While contact lenses and eyeglasses can compensate for some ailments, ophthalmic surgery may be required for others. In some instances, implants may be beneficial or desirable. For example, an intraocular lens may replace a clouded natural lens within an eye to improve vision.

While the benefits of intraocular lenses and other implants are known, improvements to delivery systems, components, and processes continue to improve outcomes and benefit patients.

### BRIEF SUMMARY

New and useful systems, apparatuses, and methods for eye surgery are set forth in the appended claims. Illustrative embodiments are also provided to enable a person skilled in the art to make and use the claimed subject matter.

For example, some embodiments may be used to fold and compress an intraocular lens or other optical implant before inserting it into the eye in various locations using variable surgical techniques. Such embodiments may comprise or consist essentially of a haptic folding mechanism configured to fold one or more haptics onto the top of an optic prior to the optic being folded into a nozzle. In some embodiments, a lifting mechanism can be configured to raise and constrain a leading haptic during implant delivery. This mechanism can constrain the leading haptic higher than the optic body so the leading haptic can passively drop down onto the top of the optic body.

In more particular examples, a haptic lifter can raise a leading haptic so that the leading haptic is higher than the optic body in an initial configuration. The lens can be pushed forward during delivery, which advances the optic body beneath the leading haptic. The haptic lifter can initially constrain the leading haptic, which can then be folded and released on top of the optic body while the optic body continues to move forward.

More generally, some embodiments of an apparatus for eye surgery may comprise an implant bay comprising a haptic lifter and an implant disposed in the implant bay. The implant may comprise an optic body and a leading haptic, and the haptic lifter can elevate the leading haptic relative to the optic body. In more particular embodiments, the optic body can be configured to be advanced under the leading haptic, and the haptic lifter can be configured to retain the leading haptic until the optic body is under the leading haptic. In some embodiments, the optic body has a leading edge and a trailing edge, and the implant bay can elevate the leading edge relative to the trailing edge.

More particular embodiments may additionally comprise a plunger configured to advance the optic body under the leading haptic while the haptic lifter retains a distal end of the leading haptic over the optic body. In some embodiments, the plunger can be configured to advance the optic body until the distal end of the leading haptic is released from the haptic lifter and falls onto the optic body.

In other aspects, an apparatus for eye surgery may comprise a nozzle; an implant bay coupled to the nozzle, the implant bay comprising a base, a cap coupled to the base to form a cavity within the implant bay, and a haptic lifter; an implant disposed in the cavity, the implant comprising an optic body, a leading haptic, and a trailing haptic; and an actuator coupled to the base. The haptic lifter can elevate the leading haptic relative to the optic body and the actuator can be configured to fold the trailing haptic onto the optic body and advance the optic body under the leading haptic toward the nozzle until the leading haptic is released from the haptic lifter onto the optic body. The actuator can be configured to advance the implant from the implant bay into the nozzle with the leading haptic and the trailing haptic on the optic body.

In yet other example embodiments, an apparatus for eye surgery may comprise a nozzle having a delivery lumen, an implant bay, an implant, and an actuator. The implant bay may comprise a base coupled to the nozzle and a cap coupled to the base to form a cavity within the implant bay that is fluidly coupled to the delivery lumen. The base may comprise a haptic lifter and an optic ramp. The implant may be disposed in the cavity. In some embodiments, the implant may comprise an optic body having a leading edge and a trailing edge, and the optic body may be disposed on the optic ramp so that the leading edge is elevated relative to the trailing edge. The implant may further comprise a leading haptic and a trailing haptic coupled to the optic body. The leading haptic may have a distal end constrained by the haptic lifter so that the distal end is elevated relative to the leading edge. The actuator may be coupled to the base and may comprise a housing and a plunger at least partially disposed within the housing. The plunger can be configured to fold the trailing haptic onto the optic body, advance the optic body under the leading haptic toward the delivery lumen until the leading haptic is released from the haptic lifter onto the optic body, and advance the implant from the implant bay through the delivery lumen with the leading haptic and the trailing haptic on the optic body.

Features, elements, and aspects described in the context of some embodiments may also be omitted, combined, or replaced by alternative features. Other features, objectives, advantages, and a preferred mode of making and using the claimed subject matter are described in greater detail below with reference to the accompanying drawings of illustrative embodiments.

### BRIEF DESCRIPTION OF THE DRAWINGS

The accompanying drawings illustrate some objectives, advantages, and a preferred mode of making and using some embodiments of the claimed subject matter. Like reference numbers represent like parts in the examples.

FIG. 1 is an isometric view of an example apparatus for delivering an implant into an eye.

FIG. 2 is an exploded view of the apparatus of FIG. 1.

FIG. 3 is a top view of a base and an implant associated with the apparatus of FIG. 2.

FIG. 4 is a front view of the base and the implant of FIG. 3.

FIG. 5A is a partial section view of the apparatus of FIG. 1, illustrating the apparatus in a first state.

FIG. 5B is a partial section view of the apparatus of FIG. 1, illustrating the apparatus in a second state.

FIG. 5C is a partial section view of the apparatus of FIG. 1, illustrating the apparatus in a third state.

FIG. 5D is a partial section view of the apparatus of FIG. 1, illustrating the apparatus in a fourth state.

FIG. 6A and FIG. 6B are schematic diagrams illustrating an example use of the apparatus of FIG. 1 to deliver an implant to an eye.

### DESCRIPTION OF EXAMPLE EMBODIMENTS

The following description of example embodiments provides information that enables a person skilled in the art to make and use the subject matter set forth in the appended claims, but it may omit certain details already well known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive an implant. However, as should be recognized by those skilled in the art, this frame of reference is merely a descriptive expedient rather than a strict prescription.

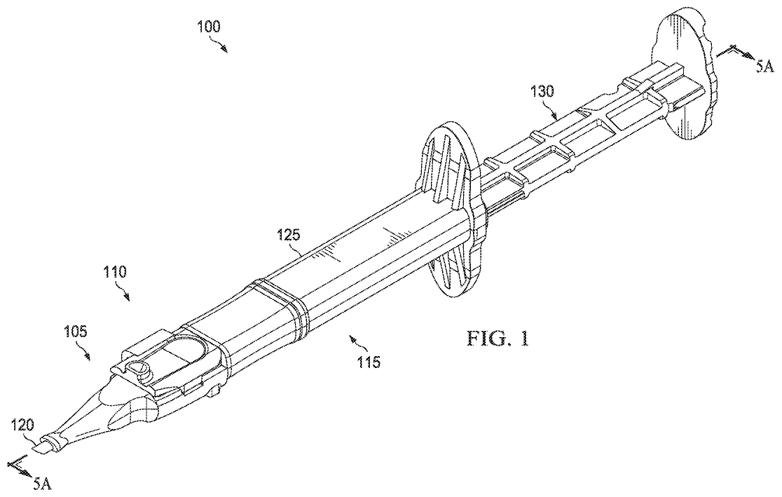

FIG. 1 is an isometric view of an example of an apparatus 100 that can deliver an implant into an eye. In some embodiments, the apparatus 100 may comprise two or more modules, which can be configured to be coupled and decoupled as appropriate for storage, assembly, use, and disposal. As illustrated in FIG. 1, some embodiments of the apparatus 100 may include a nozzle 105, an implant bay 110 coupled to the nozzle 105, and an actuator 115 coupled to the implant bay 110.

The nozzle 105 generally comprises a tip 120 adapted for insertion through an incision into an eye. The size of the tip 120 may be adapted to surgical requirements and techniques as needed. For example, small incisions are generally preferable to reduce or minimize healing times. Incisions of less than 2 millimeters may be preferable in some instances, and the tip 120 of the nozzle 105 may have a width of less than 2 millimeters in some embodiments.

The implant bay 110 generally represents a wide variety of apparatuses that are suitable for storing an implant prior to delivery into an eye. In some embodiments, the implant bay 110 may additionally or alternatively be configured to prepare an implant for delivery. For example, some embodiments of the implant bay 110 may be configured to be actuated by a surgeon or other operator to prepare an implant for delivery by subsequent action of the actuator 115. In some instances, the implant bay 110 may be configured to actively deform, elongate, extend, or otherwise manipulate features of the implant before the implant is advanced into the nozzle 105. For example, the implant bay 110 may be configured to fold, tuck, extend or splay one or more features, such as haptics, of an intraocular lens.

The actuator 115 is generally configured to advance an implant from the implant bay 110 into the nozzle 105, and thereafter from the nozzle 105 through an incision and into an eye. The actuator 115 of FIG. 1 generally comprises a housing 125 and a plunger assembly 130. The housing 125 and the plunger assembly 130 are generally comprised of a substantially rigid material, such as a medical grade polymer material.

In general, components of the apparatus 100 may be coupled directly or indirectly. For example, the nozzle 105 may be directly coupled to the implant bay 110 and may be indirectly coupled to the actuator 115 through the implant bay 110. Coupling may include fluid, mechanical, thermal, electrical, or chemical coupling (such as a chemical bond), or some combination of coupling in some contexts. For example, the implant bay 110 may be mechanically coupled to the actuator 115 and may be mechanically and fluidly coupled to the nozzle 105. In some embodiments, components may also be coupled by virtue of physical proximity, being integral to a single structure, or being formed from the same piece of material.

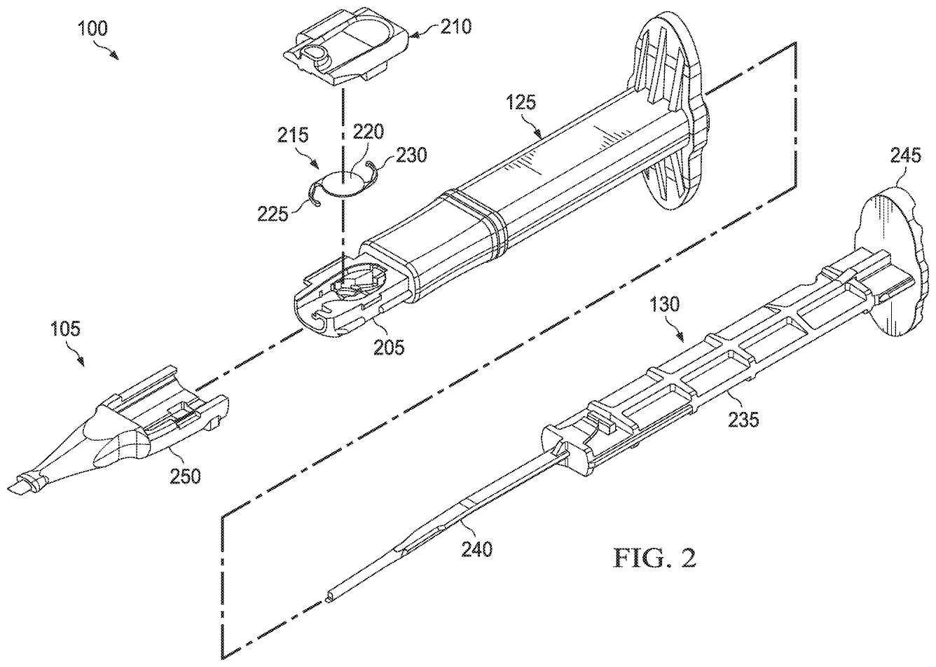

FIG. 2 is an exploded view of the apparatus 100 of FIG. 1, illustrating additional details that may be associated with some embodiments. For example, the implant bay 110 (see FIG. 1) of FIG. 2 comprises a base 205 and a cap 210, which may be coupled to the base 205 as shown in FIG. 1. The base 205 may extend from or be coupled to the housing 125 in some examples.

An implant 215 may be disposed between the base 205 and the cap 210. In the example of FIG. 2, the implant 215 is an intraocular lens having an optic body 220. In some examples, the optic body 220 may have a shape similar to that of a natural lens of an eye. Examples of suitable materials may include silicone, acrylic, and combinations of such suitable materials. The implant 215 may also comprise one or more features for positioning the optic body 220 within an eye, such as a leading haptic 225 and a trailing haptic 230. In the example of FIG. 2, the leading haptic 225 and the trailing haptic 230 extend from opposing sides of the optic body 220. In some instances, the implant 215 may be filled with a fluid, such as a fluid-filled accommodating intraocular lens.

As shown in the example of FIG. 2, some embodiments of the plunger assembly 130 may comprise a push rod 235, a plunger 240 coupled to one end of the push rod 235, and a finger flange 245 coupled to the opposite end of the push rod 235.

The nozzle 105 of FIG. 2 comprises a bay interface 250, which can be coupled to the base 205.

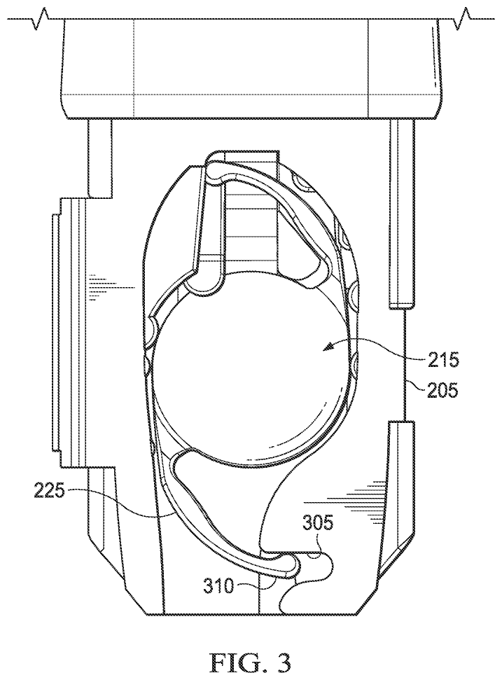

FIG. 3 is a top view of the base 205 and the implant 215 of FIG. 2, illustrating additional details that may be associated with some embodiments. The base 205 of FIG. 3 comprises a haptic lifter 305, which is configured to receive a portion of the leading haptic 225. As shown in the example of FIG. 3, some embodiments of the haptic lifter 305 may comprise a pocket, notch, groove, recess, catch, or similar feature, which may be integral to the base 205. The haptic lifter 305 may be configured to receive a distal end 310 of the leading haptic 225.

FIG. 4 is a front view of the base 205 and the implant 215 of FIG. 3, illustrating additional details that may be associated with some embodiments. As illustrated in the example of FIG. 4, the optic body 220 can recessed within the base 205 and the haptic lifter 305 can elevate a portion of the leading haptic 225 relative to the optic body 220. In more particular examples, the haptic lifter 305 can elevate a portion of the leading haptic 225 relative to a leading edge 405 of the optic body 220.

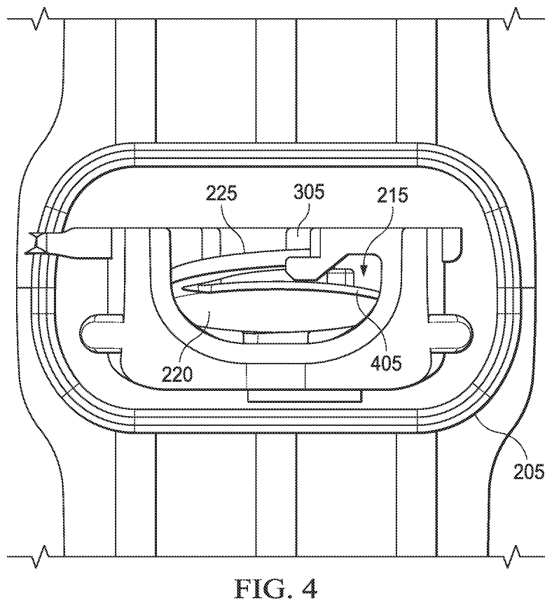

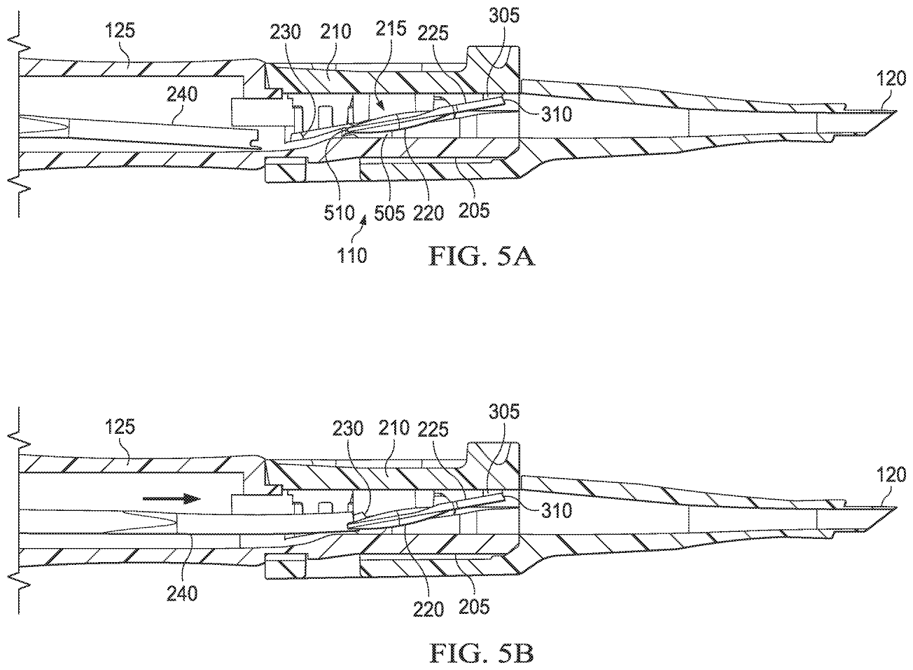

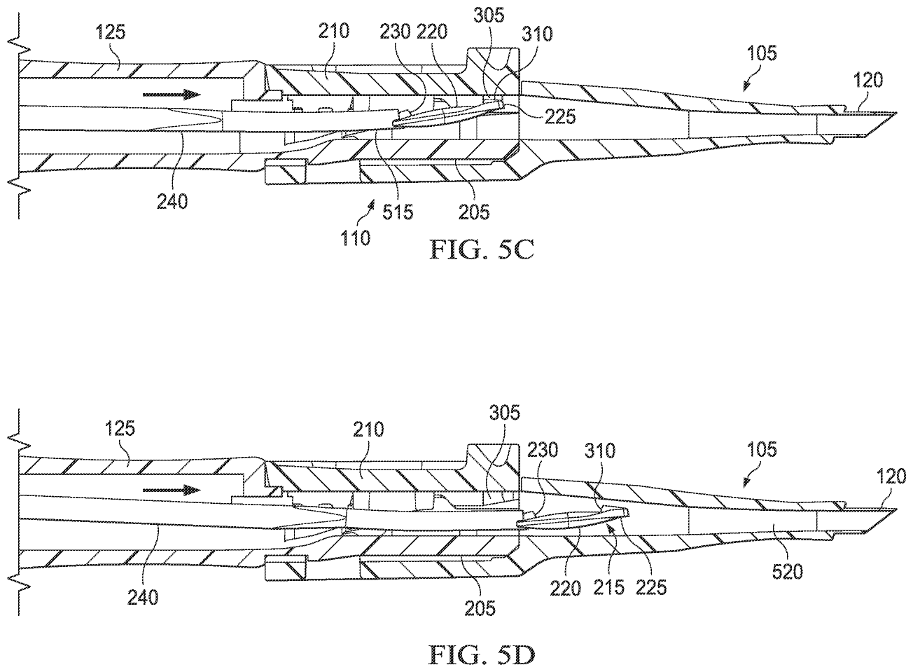

FIGS. 5A-5D are section views of the apparatus 100 of FIG. 1, illustrating additional details that may be associated with some embodiments in various states of operation. For example, FIG. 5A illustrates the apparatus 100 in a first state, in which the implant 215 is disposed within the implant bay 110. More particularly, as illustrated in FIG. 5A, the cap 210 may be coupled to the base 205, forming a cavity in the implant bay 110 between the cap 210 and the base 205, and the implant 215 may be disposed within the cavity. In some embodiments, the optic body 220 may be tilted within the base 205. For example, an optic ramp 505 can support at least a portion of the optic body 220 so that the leading edge 405 (see FIG. 4) is elevated relative to the trailing edge 510, while the distal end 310 of the leading haptic 225 remains elevated relative to the optic body 220 (and more particularly, relative to the leading edge 405).

FIG. 5B illustrates additional details that may be associated with the example of FIG. 5A in a second state. With the distal end 310 of the leading haptic 225 retained in the haptic lifter 305 and the leading haptic 225 lifted relative to the optic body 220, the plunger 240 can be advanced toward the implant 215 to fold the trailing haptic 230 over the optic body 220, as illustrated in FIG. 5B.

FIG. 5C illustrates additional details that may be associated with the example of FIG. 5A in a third state. In some embodiments, the optic body 220 can be advanced under the leading haptic 225. For example, the plunger 240 may be configured to advance the optic body 220 under the leading haptic 225 while the haptic lifter 305 retains the distal end 310 in an elevated configuration. In more particular examples, a tip 515 of the plunger 240 may be advanced through the implant bay 110, with the leading haptic 225 lifted and the trailing haptic 230 folded as shown in FIG. 5B. As the plunger 240 is advanced, the tip 515 can advance the optic body 220 toward the nozzle 105 and under the leading haptic 225.

FIG. 5D illustrates additional details that may be associated with the example of FIG. 5A in a fourth state. In the example of FIG. 5D, the optic body 220 can be advanced by the plunger 240 until the leading haptic 225 is released from the haptic lifter 305 and falls onto the top of the optic body 220. For example, in some embodiments, advancement of the optic body 220 can create tension in the leading haptic 225 until the distal end 310 of the leading haptic 225 is pulled from the haptic lifter 305 onto the optic body 220. The plunger 240 can be advanced further to move the implant 215 in the folded configuration through a delivery lumen 520 in the nozzle 105 until the implant 215 is ejected through the tip 120.

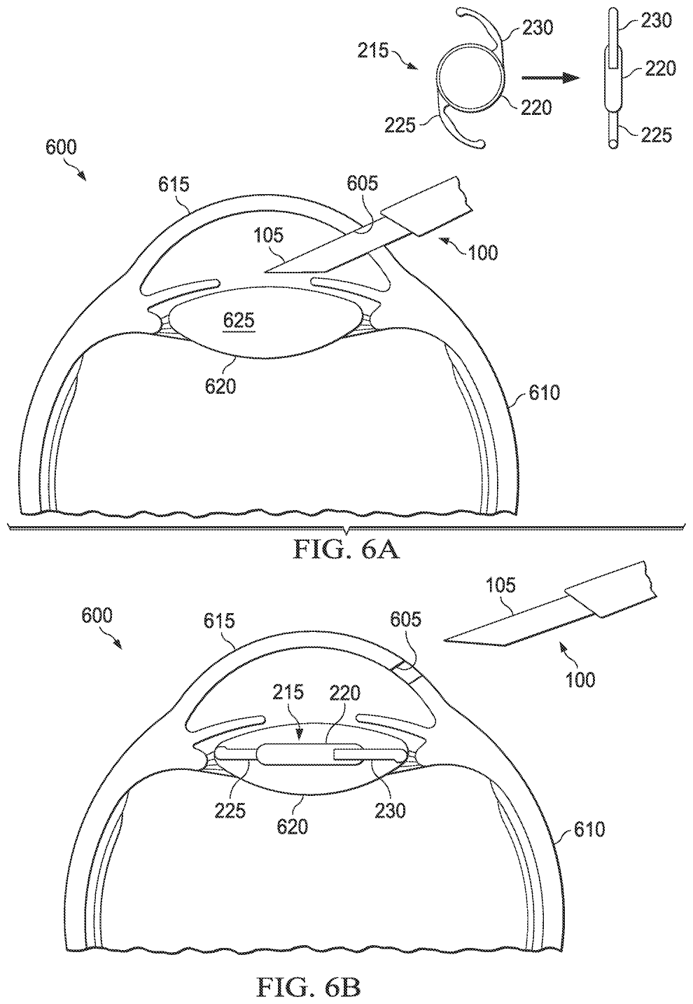

FIG. 6A and FIG. 6B are schematic diagrams illustrating an example use of the apparatus 100 of FIG. 1 to deliver the implant 215 to an eye 600. As illustrated, an incision 605 may be made in the eye 600 by a surgeon, for example. In some instances, the incision 605 may be made through the sclera 610 of the eye 600. In other instances, an incision may be formed in the cornea 615 of the eye 600. The incision 605 may be sized to permit insertion of a portion of the nozzle 105 to deliver the implant 215 into the capsular bag 620. For example, in some instances, the size of the incision 605 may have a length less than about 3000 microns (3 millimeters). In other instances, the incision 605 may have a length of from about 1000 microns to about 1500 microns, from about 1500 microns to about 2000 microns, from about 2000 microns to about 2500 microns, or from about 2500 microns to about 3000 microns.

After the incision 605 is made, the nozzle 105 can be inserted through the incision 605 so that the width of the tip 120 aligns with the length of the incision 605, allowing the nozzle 105 to extend into an interior portion 625 of the eye 600. The apparatus 100 can then eject the implant 215 through the nozzle 105 into the capsular bag 620 of the eye 600, substantially as described with reference to FIG. 5A to FIG. 5D.

In some embodiments, the implant 215 may comprise an intraocular lens having a shape similar to that of a natural lens of an eye, and it may be made from numerous materials. Examples of suitable materials may include silicone, acrylic, and combinations of such suitable materials. In some instances, the implant 215 may comprise an intraocular lens that is fluid-filled, such as a fluid-filled accommodating intraocular lens.

The implant 215 may be delivered in a folded configuration and can revert to a resting state with the leading haptic 225 and the trailing haptic 230 being at least partially curved around the optic body 220, within the capsular bag 620, as shown in FIG. 5B. The capsular bag 620 can retain the implant 215 within the capsular bag 620 in a relationship relative to the eye 600 so that the optic body 220 refracts light directed to the retina (not shown). The leading haptic 225 and the trailing haptic 230 can engage the capsular bag 620 to secure the implant 215 therein. After delivering the implant 215 into the capsular bag 620, the nozzle 105 may be removed from the eye 600 through the incision 605, and the eye 600 can be allowed to heal over time.

The systems, apparatuses, and methods described herein may provide significant advantages. For example, some embodiments may be particularly advantageous for delivering intraocular implants. More particular advantages of some embodiments may include providing high-consistency folding of leading haptics without significantly increasing complexity or cost. Consistent and reliable haptic folding can significantly increase consistency and reliability for implant delivery through small incisions across the diopter range, particularly with a pre-loaded implant delivery system.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations, the nozzle 105, the implant bay 110, and the actuator 115 may each be separated from one another or combined in various ways for manufacture or sale.

The claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

What is claimed is:

1. An apparatus for eye surgery, the apparatus comprising:
an implant bay comprising a haptic lifter; and
an implant disposed in the implant bay, the implant comprising an optic body and a leading haptic;
wherein the haptic lifter elevates the leading haptic relative to the optic body.

2. The apparatus of claim 1, wherein:
the optic body is configured to be advanced under the leading haptic; and
the haptic lifter is configured to retain the leading haptic until the optic body is under the leading haptic.

3. The apparatus of claim 1, wherein the haptic lifter comprises a pocket in the implant bay.

4. The apparatus of claim 1, wherein the haptic lifter comprises a pocket in the implant bay that is elevated relative to the optic body.

5. The apparatus of claim 1, wherein:
the optic body has a leading edge and a trailing edge; and
the implant bay elevates the leading edge relative to the trailing edge.

6. The apparatus of claim 1, further comprising a plunger configured to advance the optic body under the leading haptic while the haptic lifter retains the leading haptic.

7. The apparatus of claim 6, wherein the plunger is configured to advance the optic body until the leading haptic is released from the haptic lifter and falls onto the optic body.

8. The apparatus of claim 6, wherein:
the implant further comprises a trailing haptic; and
the plunger is further configured to fold the trailing haptic over the optic body.

9. The apparatus of claim 8, wherein the plunger is configured to fold the trailing haptic over the optic body before advancing the optic body under the leading haptic.

10. An apparatus for eye surgery, the apparatus comprising:
a nozzle;
an implant bay coupled to the nozzle, the implant bay comprising a base, a cap coupled to the base to form a cavity within the implant bay, and a haptic lifter;
an implant disposed in the cavity, the implant comprising an optic body, a leading haptic, and a trailing haptic; and
an actuator coupled to the base;
wherein the haptic lifter elevates the leading haptic relative to the optic body and the actuator is configured to fold the trailing haptic onto the optic body and advance the optic body under the leading haptic toward the nozzle until the leading haptic is released from the haptic lifter onto the optic body.

11. The apparatus of claim 10, wherein the actuator is further configured to advance the implant from the implant bay into the nozzle with the leading haptic and the trailing haptic on the optic body.

12. The apparatus of claim 10, wherein the haptic lifter comprises a pocket in the implant bay.

13. The apparatus of claim 10, wherein the haptic lifter comprises a pocket in the implant bay that is elevated relative to the optic body.

14. The apparatus of claim 10, wherein:
the optic body has a leading edge and a trailing edge; and
the implant bay elevates the leading edge relative to the trailing edge.

15. An apparatus for eye surgery, the apparatus comprising:
a nozzle having a delivery lumen;
an implant bay comprising:
a base coupled to the nozzle, the base comprising a haptic lifter and an optic ramp, and
a cap coupled to the base to form a cavity within the implant bay that is fluidly coupled to the delivery lumen;
an implant disposed in the cavity, the implant comprising:
an optic body having a leading edge and a trailing edge, the optic body disposed on the optic ramp so that the leading edge is elevated relative to the trailing edge,
a leading haptic having a distal end constrained by the haptic lifter so that the leading haptic is elevated relative to the leading edge, and
a trailing haptic; and
an actuator coupled to the base, the actuator comprising:
a housing, and
a plunger at least partially disposed within the housing;
wherein the plunger is configured to fold the trailing haptic onto the optic body, advance the optic body under the leading haptic toward the delivery lumen until the leading haptic is released from the haptic lifter onto the optic body, and advance the implant from the implant bay through the delivery lumen with the leading haptic and the trailing haptic on the optic body.
